(19) United States
(12) Patent Application Publication
(10) Pub. No.: US 2017/0197018 A1
(43) Pub. Date: Jul. 13, 2017

(54) MOTORIZED CHEST DRAINAGE SYSTEM

(71) Applicant: Covidien LP, Mansfield, MA (US)

(72) Inventors: Nilay Mukherjee, Burlington, MA (US); Sachin Shah, Milford, CT (US); David Racenet, Killingworth, CT (US)

(21) Appl. No.: 15/378,532

(22) Filed: Dec. 14, 2016

Related U.S. Application Data
(60) Provisional application No. 62/275,829, filed on Jan. 7, 2016.

(51) Int. Cl. A61M 1/00

(52) U.S. Cl. CPC A61M 1/0082; A61M 1/0027; A61M 1/0029; A61M 1/0049; A61M 1/0084; A61M 2205/3334; A61M 2205/502; A61M 2205/8206; A61M 2205/3576

(57) ABSTRACT

A chest drainage system is disclosed, including a flexible tube with an articulable tip and a control assembly. The control assembly is operated by a motor and includes an actuation assembly. The actuation assembly is operatively coupled with the flexible tube and is adapted to articulate the tip.

### CROSS-REFERENCE TO RELATED APPLICATIONS

This application claims the benefit of and priority to U.S. Provisional Patent Application No. 62/275,829 filed Jan. 7, 2016, the entire disclosure of which is incorporated by reference herein.

### TECHNICAL FIELD

The present disclosure relates generally to a medical drainage system, and in particular, a motorized chest drainage system and a method of use thereof.

### BACKGROUND

The human pleura and chest wall are normally in opposition. A variety of iatrogenic or organic states can least to interposition of either gas or fluid between the pleura and chest wall, which if unchecked may compromise the lungs and may cause a variety of sequelae, from shortness of breath to profound hypoxia/hypotension. Thoracic surgical procedures where the pleura are violated may naturally result in pneumothorax; also, occasionally people may spontaneously pop diseased lung tissue, which also results in pneumothorax. In lung resection procedures, this problem can persist, therefore chest drainage systems are routinely placed within a patient's pleural space to treat this pneumothorax and minimize the accumulation of fluid. In certain disease states including trauma, pneumonia, and cancer, fluid may accumulate in the pleural space more quickly than it can be evacuated. As noted, this may lead to unchecked compression of the lungs leading to the aforementioned sequelae. Frequently these fluid collections move. A chest drainage system with a motorized tip may assist in the removal of fluid within a patient's pleural cavity.

A basic chest drainage system includes a chest tube and drainage canister. Advancements have been made to the basic system. A chest drainage system can now include a suction system, de-clogging system, or a sensor system or any combination thereof. The inclusion of these systems permits a chest drainage system to appropriately handle the dynamic atmosphere within the pleural cavity. However, despite these advancements, chest drainage systems may still lack mobility once placed within a patient's pleural cavity.

Accordingly, a motorized chest drainage system capable of being repositioned after being placed within the patient's pleural cavity is desirable.

### SUMMARY

The present disclosure is directed to a motorized chest drainage system. The motorized chest drainage system includes a flexible tube having proximal and distal ends, a tip positioned at the distal end, an articulation assembly operatively coupled with the flexible tube, and a control assembly which is operated by a motor. The control assembly is operatively coupled with the articulation assembly, and the articulation assembly is adapted to articulate the tip.

In one embodiment, the chest drainage system may include a suction source. This suction source may be coupled with a sensor unit that will collect data on the suction pressure, fluid flow rate, or content type or any combination thereof. The sensor may also be coupled with a data processor, which will evaluate the data collected by the sensor. The data evaluated by the data processor may be communicated to a display located on the case of the device. The case may house the articulation assembly, the motorized control assembly, the suction source, sensor, and data processor. The case may also include controls for the articulation assembly, a motor control, a power outlet or battery, or both, and a fluid reservoir that will collect all fluids being drained from a patient's pleural cavity.

In another embodiment, there may be an optionally detachable sensor unit that will attach in-line with the chest drainage tube, between the distal end of the chest tube and the case of the drainage system. The detachable sensor unit will connect to the chest tube via a set of fittings at each end of the detachable sensor unit. The detachable sensor unit might contain one or more sensors that could monitor various parameters of the fluids and/or solids that travel through the detachable sensor unit. These parameters include, but are not limited to, pressure, flow rate, pH, presence of blood, carbon dioxide levels, glucose levels, as well as other parameters of the contents of the chest tube. The detachable sensor unit may also contain wireless communications capabilities. The detachable sensor unit may also contain a data processor, which would analyze the data collected by the various sensors in the detachable sensor unit, and wirelessly communicate information about the analyzed data to mobile communication devices, such as mobile phones or pagers carried by monitoring personnel. Additionally, the detachable sensor unit may have a display system, which can display data collected by the various sensors. The detachable sensor unit may also contain a replaceable battery to provide power for the sensors, data processor, display system, and wireless communications units.

In another embodiment, the articulation assembly may be remotely operated, programmed to have a set oscillation pattern, programmed to have a user defined pattern or any combination thereof.

These and other features of the current disclosure will be explained in greater detail in the following detailed descriptions of the various embodiments.

### BRIEF DESCRIPTION OF THE DRAWINGS

Various embodiments of the present disclosure are described herein below with reference to the drawings, wherein:

FIG. 1 is a perspective view of a motorized chest drainage system in accordance with the present disclosure;

FIG. 2 is an interior view of a motorized chest drainage system as shown in FIG. 1;

FIG. 3 is a rear perspective view of a control assembly, including multiple connecting members;

FIG. 4 is an enlarged area of detail view of the first and second segments as shown in FIG. 1, showing bi-directional articulation of the first and second segments in a first plane and a second plane;

FIG. 5 is a perspective view of the control assembly shown in FIG. 3 positioned within an outer housing and coupled to a motor;

FIG. 6 is a basic schematic design for the display system; and

FIG. 7 is a perspective view of a detachable sensor unit;

FIG. 8 is an interior view of the detachable sensor unit as shown in FIG. 7; and

FIG. 9 is a basic schematic design for the display system within the sensor unit.

Other features of the present disclosure will become apparent from the following detailed description taken in conjunction with the accompanying drawings, which illustrate, by way of example, the principles of the present disclosure.

### DETAILED DESCRIPTION

Embodiments of the presently disclosed motorized chest drainage system are described in detail with reference to the drawings, in which like reference numerals designate identical or corresponding elements in each of the several views. As used herein, the term “clinician” refers to a doctor, a nurse, or any other care provider. Throughout this description, the term “distal” refers to that portion of the tool or component thereof which is farther from the user while the term “proximal” refers to the portion of the tool or component thereof which is closer to the user. The presently disclosed chest drainage system is usable in openings through a patient's tissue.

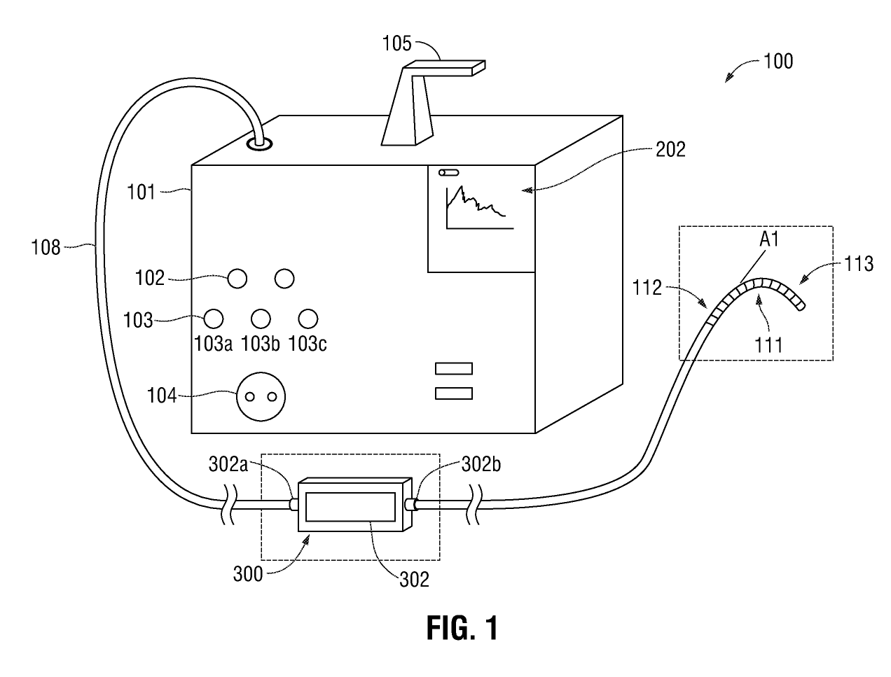

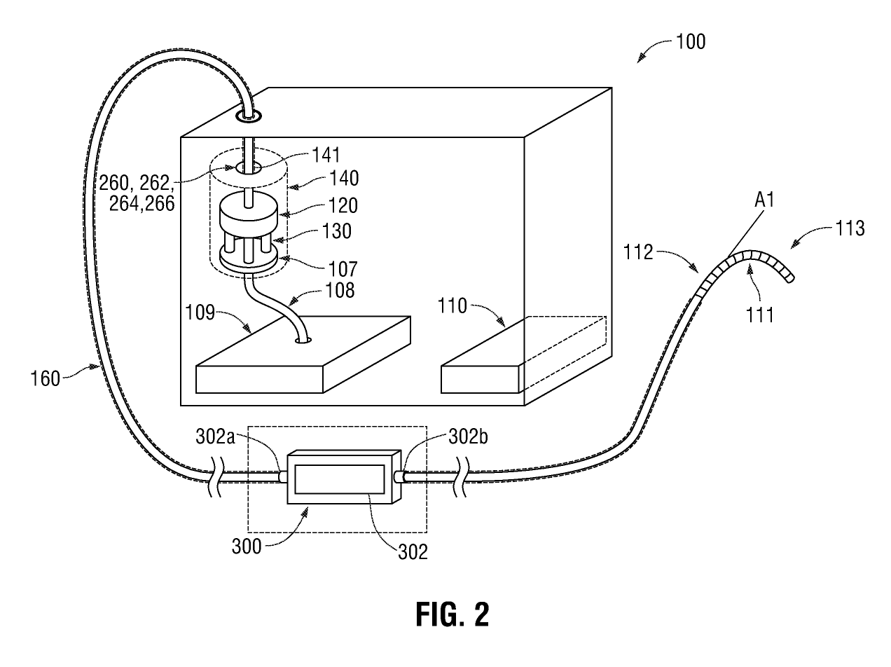

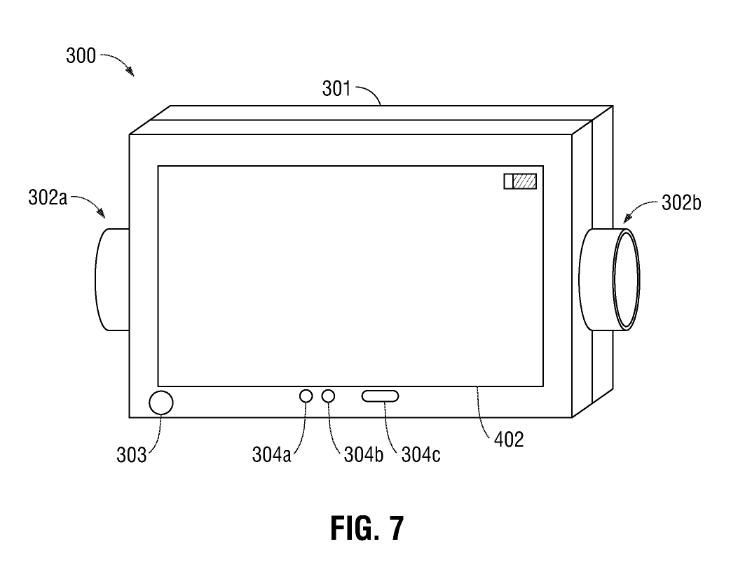

Referring initially to FIG. 1, a motorized chest drainage system 100 is shown. Motorized chest drainage system 100 includes a case 101, which houses select components of the chest drainage system 100. The case 101 includes actuation controls 102 that permit a user to operate an actuation assembly 130 (FIG. 2), motor controls 103 that allow the user to choose between remotely controlling the actuation assembly 130 or a set oscillation pattern or a user defined pattern, and a display 202, all of which will be described in further detail below. Also, the case 101 includes an outlet 104 and a handle 105. Extending from the case 101 is a chest tube 108 having an articulable tip 111. The motorized chest drainage system 100 may include an optional, detachable sensor unit 300. Sensor unit 300 is shown connected in-line with the chest tube 108. With additional reference to FIG. 7, the sensor unit 300 includes a set of fittings 302a and 302b at each end to allow attachment to the chest drainage tube 108. The fittings 302a and 302b may be made of a suitable biocompatible material such as plastic or metal. The fittings 302a and 302b may be adhered, welded, snap-fit, press fit, or otherwise coupled to the sensor 300 unit. These fittings 302a and 302b allow chest tube 108 to be removably coupled to sensor 300.

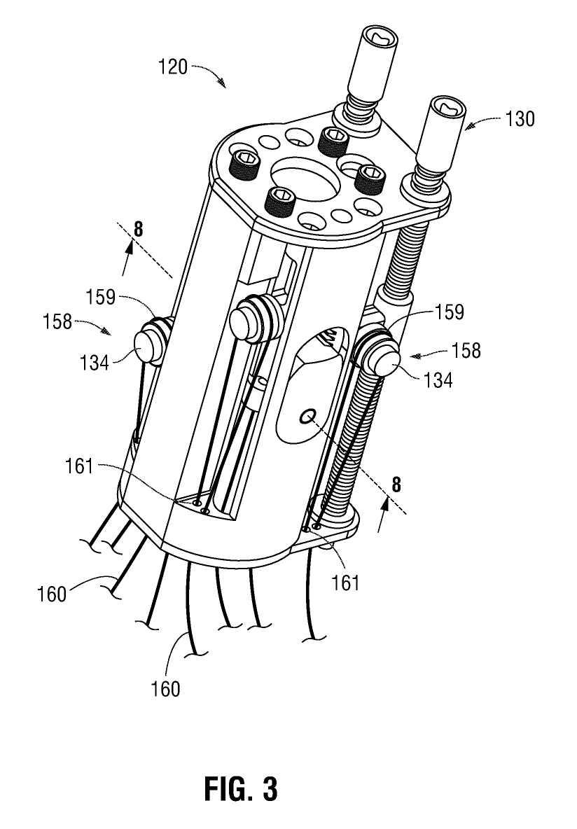

Referring now to FIG. 2, the motorized chest drainage system 100 is shown in further detail. The front plate of the case 101 is removed to show a fluid reservoir 109, a battery 110, and a housing 140 (shown in phantom view). The housing 140 contains a control assembly 120, an articulation assembly 130, sensors 260, 262, 264, and 266 and a motor 107. Housing 140 may have any suitable shape to accommodate control assembly 120, articulation assembly 130, sensors 260, 262, 264, and 266 and motor 107 and includes an aperture to receive one end of the chest tube 108. Also, housing 140 includes connecting apertures 141 to receive connecting members 160 (FIG. 3). The fluid reservoir 109 is removable so that collected fluids may be properly disposed of. Alternatively, fluid reservoir 109 may be attachable to a drain system such that any collected fluids are transferred to the drain system.

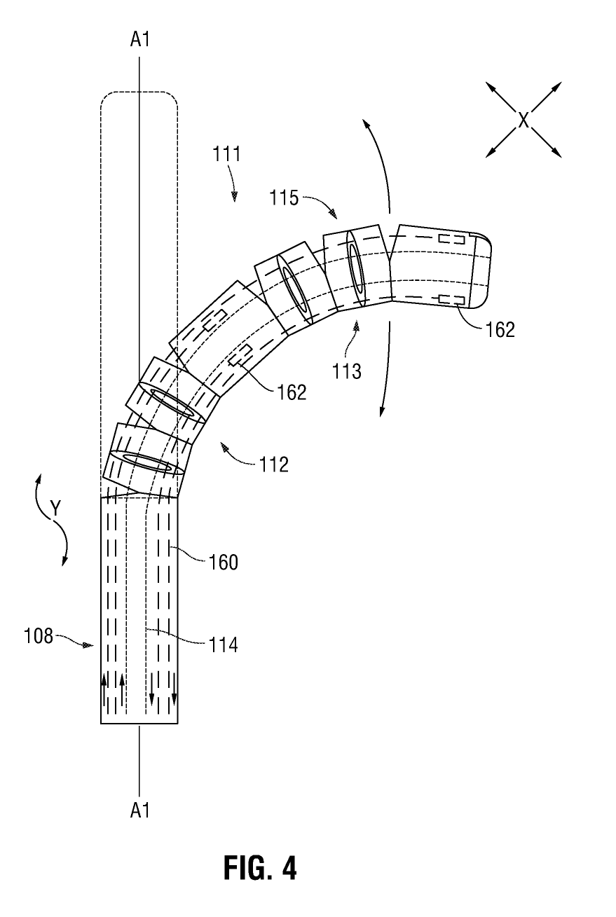

As seen in FIG. 3, control assembly 120 includes two actuation assemblies 130. Actuation assemblies 130 and associated components are substantially similar to each other, and cooperate to effect actuation of the articulable tip 111. More than two actuation assemblies 130 or less than two actuation assemblies 130 may be present in control assembly 120 to suit the particular needs of articulation. Each actuation assembly 130 is shown fully assembled, with connecting members 160 attached to portions of the actuation assembly 130. Connecting members 160 couple the actuation assemblies 130 to portions of the articulable tip 111 (FIG. 2). Connecting members 160 are shown disposed over a portion of pulleys 158 within track 159. Pulleys 158 are retained by securing members 134. Thus, when pulleys 158 rotate about securing members 134, they displace a portion of connecting members 160 disposed in the track 159. While connecting members 160 are shown as cables, connecting members 160 may be wires or other tensile elements, or may be rigid elements such as bars or links. Connecting members 160 include a proximal end 161 and a distal end 162 (FIG. 4). Ends 161, 162 of the connecting members 160 may be defined by a ferrule, or may be knotted or otherwise defined.

Referring now to FIG. 4, articulable tip 111 includes at least a first segment 112 and a second segment 113. Second segment 113 is disposed distally of the first segment 112. The first segment 112 and the second segment 113 are capable of independent movement relative to the longitudinal axis A1 and to each other. Connecting members 160 couple the actuation assemblies 130 to portions of the articulable tip 111.

As forces are transmitted to the connecting members 160 (shown in phantom view), displacement of the first and second segments 112, 113 is effected in a first plane, i.e., plane X (across the page) and a second plane, i.e., plane Y (into and out of the page). Connecting members 160 associated with a first actuation assembly 130 may be attached to opposing surfaces in each of the first and second segments 112, 113 to effect articulation in plane X, and connecting members 160 associated with a second articulation assembly 130 may be attached to opposing surfaces in each of the first and second segments 112, 113 and radially spaced from the connecting members 160 of the first articulation assembly 130 to effect articulation in plane Y. Forward and reverse engagement of the pair of actuation assemblies 130 allows for bi-directional articulation of the first and second segments 112, 113 in both plane X and plane Y. Accordingly, articulable tip 111 can be articulated in opposing directions in multiple planes. The first and second segments 112, 113 of the articulable tip 111 may be continuous flexible members, or may include independently movable members 115 that, when assembled, engage in a manner such that each movable member 115 is free to pivot relative to an adjacent movable member 115.

Chest tube 108 includes a lumen 114 (shown in phantom). Lumen 114 may be a separate tube having flexibility or flexible portions to correspond to the first and second segments 112, 113 of the articulable tip 111. The lumen 114 provides an access pathway between a distal end of the chest tube 108 and a proximal portion thereof (e.g., the distal tip and the reservoir). The lumen 114 may be used for irrigation, vacuum, suction, de-clogging, or providing instrument access into the patient's pleural cavity. Suction may be delivered by a number of different methods. In one embodiment, suction may be provided by a combined vacuum/pressure system, which will be connected to lumen 114 and chest tube 108. The combined vacuum/pressure system will provide negative pressure to lumen 114, which will allow the fluids contained within the patient's pleural cavity to be drawn out quicker. The user may reverse the pressure within the lumen 114 to de-clog chest tube 108 using positive pressure to dislodge an obstruction. In another embodiment, de-clogging can be achieved by temporarily removing chest tube 108 from the pleural cavity and manually removing the obstruction. In another embodiment, de-clogging might be achieved by a morcellator disposed inside lumen 114 that grinds up any blocking material into small enough pieces that will adequately pass through the drain and out of the patient's pleural cavity. De-clogging may also be achieved by means of an obturator to expel the blockage. In another embodiment, de-clogging might be made unnecessary by placing a filter at the distal end of the tube to prevent anything other than liquid or smaller material to pass through. Also, in another embodiment the chest tube 108 may include two or more lumens (not shown).

A detailed description of articulation assembly and methods of effecting articulation of the articulable portion are found, for example, in U.S. Patent Application Publication No. 2012-0310220, the entire contents of which is incorporated herein by reference.

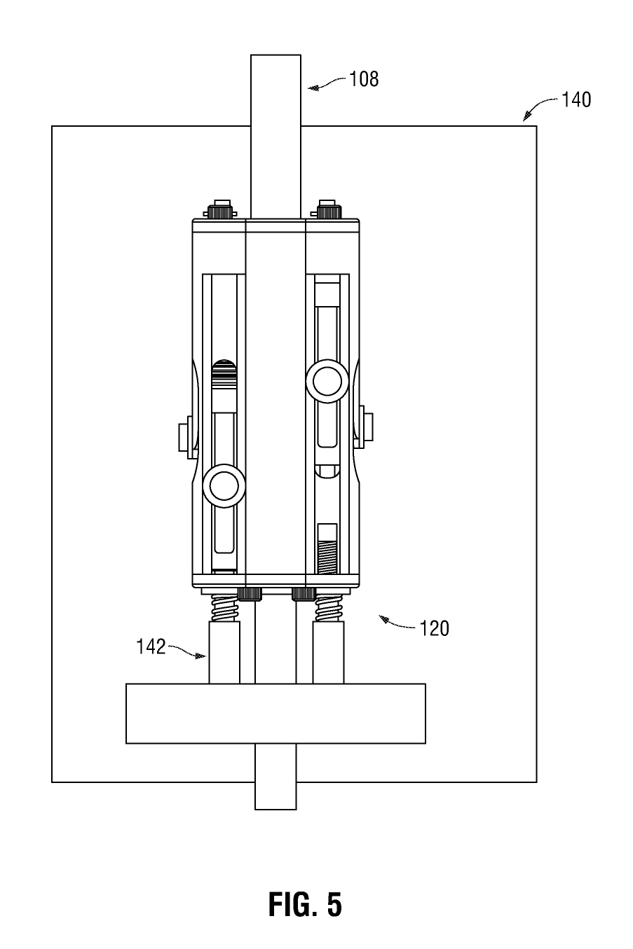

Referring to FIGS. 2 and 5, control assembly 120 is shown as placed vertically within the housing 140. The control assembly 120 may also be placed horizontally, angled or any desired position. Control assembly 120 is connected to the motor 107. The connection between the control assembly 120 and motor 107 can be achieved by a number of different connections. In one embodiment, the control assembly 120 includes drive members 141 which may have surface features to engage gear couplers (not shown) within motor 107. In another embodiment, the drive members 141 are connected to the shaft of the motor 107 via shaft couplers (not shown). The shaft couplers may be adhered, welded, snap-fit, press fit, or otherwise coupled to the drive members 141 and the motor 107. The motor 107 provides the mechanical energy necessary for the actuation assemblies 130 to transmit force to move connecting members 160 and thus articulate the articulable tip 111. Additionally, motor 107 includes an aperture to allow chest tube 108 to pass through and connect to the fluid reservoir 109 located beneath the housing 140. In another embodiment, motor 107 may be offset to avoid interference with the chest tube 108.

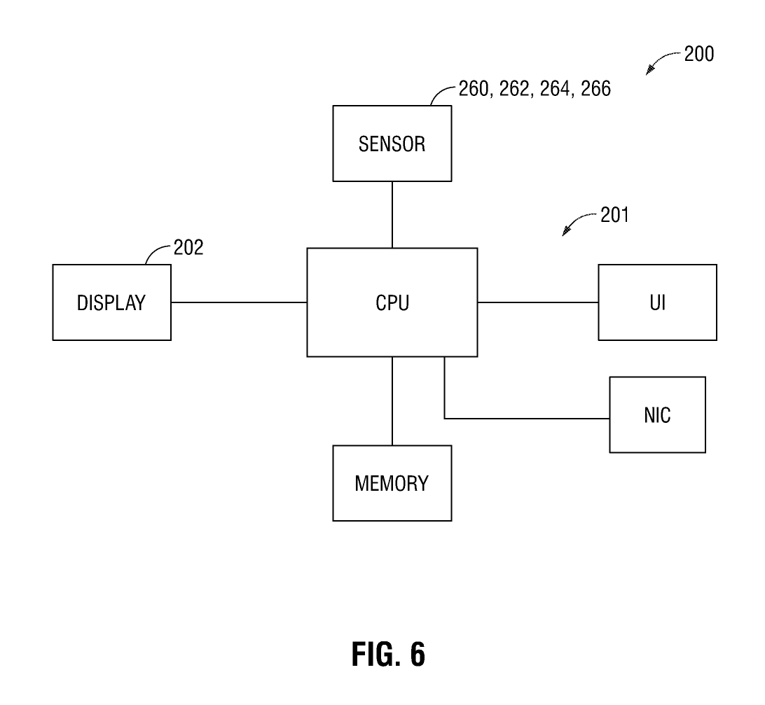

Referring additionally to FIG. 1, the actuation assemblies 130 can be remotely controlled. The user remotely controls the actuation assemblies 130 by actuation controls 102 on case 101. The actuation controls 102 will trigger motor 107 to transmit the desired force to move actuation assemblies 130, which will pull the connecting members 160 and thus articulate the articulable tip 111 in the X plane and/or Y plane (FIG. 4). The user may remotely control the actuation assemblies 130 to correct any migration of the articulable tip 111, or can articulate the articulable tip 111 between various locations to treat all pleural effusions. This will ensure the articulable tip 111 remains in the correct location within the pleural cavity at all times. Also, actuation assemblies 130 and motor 107 may be programmed to have a predefined oscillation pattern or a user defined pattern. These options may be achieved in a number of different methods. In one embodiment, the data processor (FIG. 6) may include a memory which can store an algorithm to control a set oscillation pattern, an algorithm to control a user defined pattern, a predefined program or any combination thereof. The data processor will then execute the program or algorithm stored in the memory. Both the set oscillation pattern and user defined pattern may correlate with the volume of fluid being suctioned from the patent's pleural cavity, the suction pressure, fluid flow rate, or volume of fluid contained with fluid reservoir or any combination thereof. Motor controls 103 allow the user to select between the remote control option, the set oscillation option, and the user defined option. Motor controls 103 include a plurality of user actuated buttons, knobs, or switches. A first switch 103a allows the user to select the remote control option. A second switch 103b allows the user to select the oscillation option. A third switch 103c allows the user to select the user defined option. Motor controls 103 allow the user to enter user defined pattern. In one embodiment, switches 103a, 103b and 103c will be configured so that once the desired switch is selected by the user the unselected switches will be disabled.

Turning to FIG. 6, display system 200 includes a data processor 201 and display 202. The data processor 201 may include a central processing unit, user interface, memory, sensor, and wireless component. A basic schematic of the display system 200 is shown in FIG. 6. As it is used in this description, “user interface” generally refers to any visual, graphical, tactile, audible, sensory, or other mechanism for providing information to and/or receiving information from a user or other entity. The term “user interface” as used herein may refer to an interface between a human user (or operator) and one or more devices to enable communication between the user and the device(s). Example of user interface that may be employed in various embodiments of the present disclosure include, without limitation, switches, potentiometers, buttons, dials, sliders, a mouse, a pointing device, a keyboard, a keypad, joysticks, trackballs, display screen, various types of graphical user interfaces (GUIs), touch screens, microphones and other types of sensors or devices that may receive some form of human-generated stimulus and generate a signal in response thereto. As it is used herein, “computer” generally refers to anything that transforms information in a purposeful way.

The display system 200 described herein may also utilize one or more controllers to receive various information and transform the received information to generate an output. The controller may include any type of computing device, computational circuitry, or any type of processor or processing circuitry capable of executing a series of instructions that are stored in a memory. The controller may include multiple processor and/or multicore central processing units (CPUs) and may include any type of processor, such as a microprocessor, digital signal processor, microcontroller, or the like. The controller may also include a memory to store data and/or algorithms to perform a series of instructions.

A network interface card (NIC) or other suitable network interface utilizes any known communication methods for transmitting and/or receiving data to or from sensors 260, 262, 264, and 266.

Display screen 202 (FIG. 1) may include a liquid crystal display, a light emitting diode display or the like.

Sensor 260 may be a pressure sensor for monitoring pleural pressure. Pressure sensors generate a signal related to the pressure being measured. Pressure sensors can be classified in terms of pressure ranges they measure, temperature ranges of operation, and most importantly the type of pressure they measure. In terms of pressure type, pressure sensors can be divided into five categories. Absolute pressure sensors which measure the pressure relative to perfect vacuum pressure (0 PSI or no pressure). Gauge pressure sensors may be used in different applications because it can be calibrated to measure the pressure relative to a given atmospheric pressure at a given location. Vacuum pressure sensors are used to measure pressure less than the atmospheric pressure at a given location. Sealed pressure sensors are similar to the gauge pressure sensors except that it is previously calibrated by its manufacturer to measure pressure relative to a sea level pressure. Sensor 260 may be configured to monitor pleural pressure within a predetermined range, and will trigger an alert to a clinician if the pleural pressure is outside that predetermined range. The predetermined range of sensor 260 may range between −4 and −20 cmH₂O with a sensitivity of 1 cmH₂O; however, the predetermined range of sensor 260 is not limited to this specified range, and may be greater or less. Also, a visual signal, audio signal or both may alert a clinician when the pleural pressure reaches a predetermined level, for example a clinician may be alerted when the pleural pressure equals 0 cmH₂O and/or is outside of the predetermined range by ±5 cmH₂O. In another embodiment sensor 260 may monitor fluid pressure. Additionally, a single pressure sensor can be used to measure fluid level in a container.

Sensor 260 may also be a sensor for monitoring fluid (e.g., liquid or gas) flow rate. Flow rate sensors generate a signal related to the velocity of the measured fluid. Differential pressure sensors measure the difference between two or more pressures introduced as inputs to the sensing unit. Differential pressure sensors may also be used to measure flow or level in pressurized vessels. Sensor 260 may be configured to monitor fluid flow rate within a predetermined range, and will trigger an alert to a clinician if the fluid flow rate is outside that predetermined range. The predetermined range for sensor 260 may range between 0 mL/h and 100 mL/h with a sensitivity of 10 mL; however, the predetermined range of fluid flow rate is not limited to this specified range, and may be greater or less. Also, a visual signal, audio signal or both may alert a clinician when the fluid low rate reaches a predetermined rate, for example a clinician may be alerted when the fluid flow rate equals 0 mL/h and/or when the fluid flow rate is outside of the predetermined range by ±100 mL/H.

In another embodiment, the fluid flow rate may be monitored by altering the shape the fluid reservoir 109 to a tipping bucket configuration. The tipping bucket configuration may include a central pivoting cone shaped fluid reservoir with a drainage valve that is mounted on a support device, a set of calibration screws, a magnet and a magnetic sensor. The set of calibration screws are mounted on the base of the support device and beneath the fluid reservoir, with each calibration screw positioned at one end of the fluid reservoir opposite of one another. The magnetic sensor may be located at the top of the support device and magnet may be placed at the top of the fluid reservoir adjacent to the magnetic sensor. The magnetic sensor may be a number of different types of sensors, for example the magnetic sensor may be a reed switch sensor. The cone shaped fluid reservoir is configured to the support device by a centrally located pivot at the base of the fluid reservoir. The fluid reservoir is dimension to contain a predetermined volume of fluid. After the fluid reservoir is filled with the predetermined volume, the fluid reservoir pivots about the central pivot, which allows the fluid reservoir to tip to one side draining all the fluid from the drainage valve of the fluid reservoir. When the fluid reservoir tips to one side it rests on one of the set of calibration screws. The set of calibration screws are placed under the fluid reservoir to provide stability for the fluid reservoir when in the tipped position. Also, when the fluid reservoir tips to one side the magnet moves from its original central location and passes by the magnetic sensor. The volume of fluid is tracked by the number of times the magnet passes by the magnetic sensor.

The clinician may consider the information gathered by the sensors 260 in evaluating when chest tube 108 should be removed from the patient. Also, the information gathered by the sensor 260 may indicate to a clinician that the chest tube 108 is dislodged or clogged. Further, the information gathered by the sensor 260 may also indicate to a clinician that there is excess drainage or a number of other clinical indications. In another embodiment, there will be a feedback loop between the suction source and the sensor 260. The motorized chest drainage system 100 will adjust the suction pressure based on the flow rate so as to minimize trauma to the healing tissue.

Sensor 262 may detect the pH levels of the fluid. Sensor 262 may be configured to monitor the pH levels of the fluid within a predetermined range, and will alert a clinician if the pH level is outside that predetermined range. The predetermined range for the pH level may range from 7.25 to 7.75 with a sensitivity of 0.1 pH; however, the predetermine range of the pH level is not limited to this specified range, and may be greater or less than this specified range. Also, a visual signal, audio signal or both may trigger an alert to a clinician when the pH level reaches a predetermined level within the range, for example a clinician may be alerted when the pH level is ≦7.5. A clinician may use the information gathered from sensor 262 about the pH level to determine the onset of an infection.

Sensor 264 may detect level of carbon dioxide (CO₂). Sensor 264 may be configured to monitor the level of CO₂ within a predetermined range, and will trigger an alert to a clinician if the level of CO₂ is outside that predetermined range. The predetermined range for the CO₂ level may range from 0.3 mmHg to 40 mmHg with a sensitivity of 0.1 mmHg; however, the predetermine range of the CO₂ level is not limited to this specified range, and may be greater or less than this specified range. Also, a visual signal, audio signal or both may trigger an alert to a clinician when the CO₂ level reaches a predetermined level, for example when the CO₂ level is >0 mmHg and/or >8 mmHg. A clinician may use the information gathered from sensor 264 about the CO₂ level to determine if there is any air leaking from the lung.

Sensor 266 may detect the presence of blood in the fluid within chest tube 108. Sensor 266 may be configured to monitor for the presence of blood within a predetermined range, and will trigger an alert to a clinician if the amount of blood is outside that predetermined range. The predetermined range for the amount of blood may range from 0 red blood cells (“RBC”) per mm³ to 100,000 RBC per mm³; however, the predetermine range of the amount of blood is not limited to this specified range, and may be greater or less than this specified range. Also, a visual signal, audio signal or both may alert a clinician when the amount of blood reaches a predetermined amount, for example when the amount of blood is >100,000 RBC per mm³. In some embodiments, the sensor 266 will be a RGB sensor. An RGB sensor may measure the red, green, and blue components of light with the sensitivity similar to human vision. Detection of those colors will allow the sensor 266 to gather information on whether or not blood is present within the fluids within the chest tube 108. Sensor 266 may also monitor the fluid turbidity. The color and turbidity sensed by sensor 266 may indicate a number of different clinical indications, such as:

| | Physical Appearance | | | |
|---|---|---|---|---|
| | | | | “Milky” |
| | | | | effusion - |
| | Light yellow | | | off white/ |
| | and clear | Reddish | Cloudy, thick | yellow |
| Clinical | Normal | Presence | Presence of | Chylothorax |
| Indication | appearance | of blood | microorganisms | |
| | | | and/or white blood | |
| | | | cells | |

Also, a clinician may use the information gathered by sensor 266 to determine if there is a hemorrhage or the onset of an infection.

The data collected from the sensors 260, 262, 264, and 266 may be considered by a clinician during treatment and may be used in determining when a patient is ready to be discharged. An algorithm can be developed to analyze data to determine if a patient may safely be discharged, thereby customizing the solution to the patient and potentially reducing the cost of a patient's healthcare.

In another embodiment, display system 200 may include addition sensors to gather information about a number of different clinical metrics. In one embodiment, the display system 200 may include a sensor that can detect glucose within the fluids draining from the patient. That sensor may be configured to monitor the presence of glucose within a predetermined range, and will trigger an alert to a clinician if the amount of glucose is outside that predetermined range. The predetermined range for the amount of glucose may range from 40 mg/dL to 125 mg/dL with a sensitivity of 10 mg/dL; however, the predetermine range of the amount of glucose is not limited to this specified range, and may be greater or less than this specified range. Also, a visual signal, audio signal or both may alert a clinician when the amount of glucose reaches a predetermined amount, for example a clinician may be alerted when the amount of glucose is ≦70 mg/dL. A clinician may use the information gathered about the presence of glucose to determine the onset of an infection. In another embodiment, the display system 200 may include a sensor that can detect the presence of blood hematocrit. That sensor may be configured to monitor the presence of blood hematocrit within a predetermined range, and will trigger an alert to a clinician if the amount of blood hematocrit is outside that predetermined range. The predetermined range for the amount of blood hematocrit may range from 0% to 50% of blood hematocrit with a sensitivity of 5%; however, the predetermine range of the amount of blood hematocrit is not limited to this specified range, and may be greater or less than this specified range. Also, a visual signal, audio signal or both may alert a clinician when the amount of blood hematocrit reaches a predetermined amount, for example a clinician may be alerted when the amount of blood hematocrit is <5% and/or <25%. A clinician may use the information gathered about the amount of blood hematocrit to determine if there is hemorrhaging. In another embodiment, display system 200 may include a sensor that can detect the presence of neutrophils. The information gathered by this sensor may allow a clinician to determine the onset of an infection.

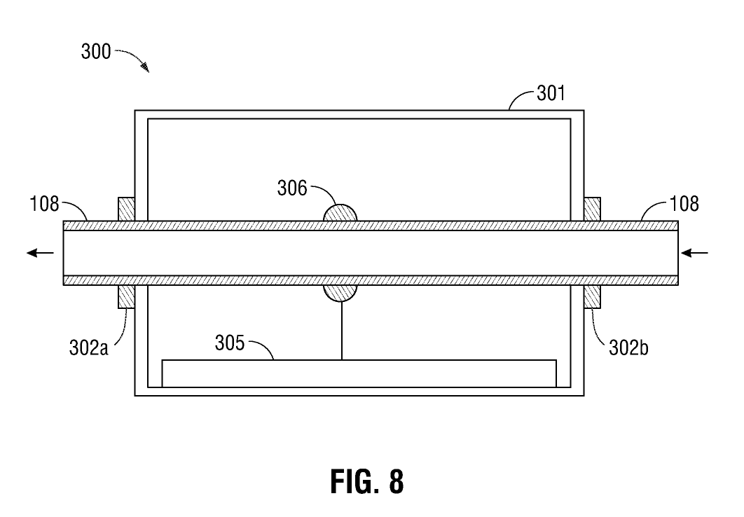

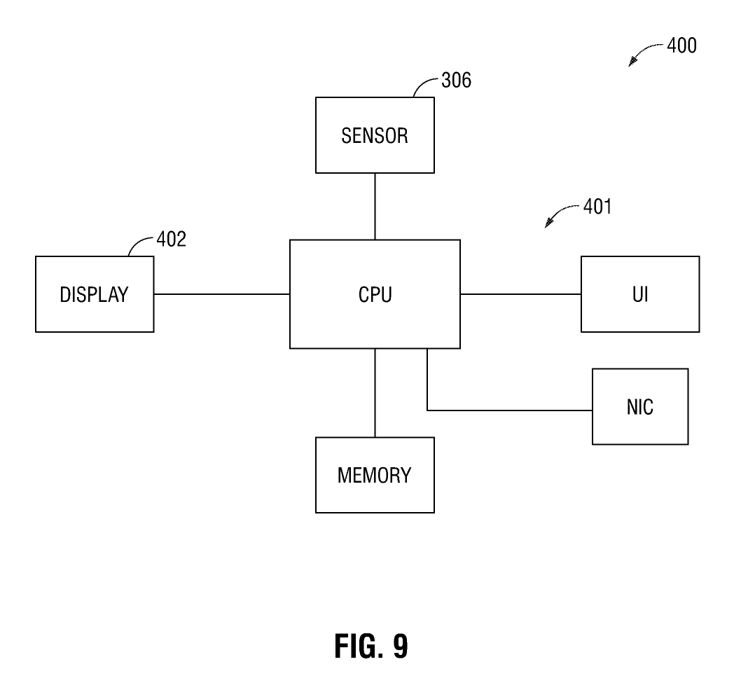

Referring now to FIGS. 7 and 8, the sensor unit 300 is shown in further detail. Senor unit 300 is shown in FIG. 7 to include a sensor case 301. In this embodiment sensor case 301 is manufactured into two individual pieces that may be adhered, welded, snap-fit, press fit, or otherwise coupled together. Also, sensor unit 300 includes a display system 400 (FIG. 9), data processor 401 (FIG. 9), display screen 402, a power button 303, two navigational buttons 304a and 304b, a selection button 304c, a battery 305 (FIG. 8) and a sensor 306 (FIG. 8). Display system 400 provides the clinician a local display of the information being gathered by sensors 260, 262, 264, and 266 and/or a local display of the information being gathered by sensor 306 (FIG. 8). Power button 303 allows the clinician to turn sensor unit 300 on, off or place it in sleep mode. Sleep mode will allow the sensor unit 300 to conserve its power by gathering information at a slower rate, only gathering pre-selected information, turning off the display while continuing to monitor parameters and collect data, or any combination thereof. Buttons 304a, 304b, and 304c are coupled to the display system 400. A clinician can select the desired option in the display system 400 by scrolling through the available options with buttons 304a and 304b, and then selecting the desired option or options with button 304c. In another embodiment, the display screen 402 may be a touch screen, which will allow the clinician to select the desired option or options by touching display screen 402. Also, in that embodiment sensor 300 can include a retractable touch pen (not shown) that would be housed in sensor case 301. The clinician can use the retractable touch pen to make the desired selection on the display screen 402 instead of using his/her finger. Additionally, sensor unit 300 may include light emitting diodes (LED's). The LED's can emit a combination of colors, such as red, yellow and green. The LED's will be coupled to display system so that the LED's will emit one of the available colors based off the status of the patient. The LEDs may indicate an alarm state for one or more particular parameters that is/are monitored by the sensors 260, 262, 264, 266, and 306. The alarm state is user programmable.

Turning to FIG. 9, display system 400 includes a data processor 401 and display screen 402. To receive various information and transform the received information to generate an output, display system 400 may utilize one or more of the controllers described above in reference to display system 200 (FIG. 6). The data processor 401 may include a central processing unit, user interface, memory, sensor, and wireless component. The data processor 401 functions the same way as data processor 201, as described above. Additionally, the user interface included in data processor 402 is the same or similar to the user interface included in the data processor 201 described above. The display screen 402 (FIG. 7) may include a liquid crystal distal, a light emitting diode display or the like. Also, the display screen 402 may be a touch screen.

A network interface car (NIC) or other suitable network interface utilizes any known communication methods for transmitting and/or receiving data to or from sensor 306.

Any of the herein described methods, programs, algorithms or codes may be converted to, or expressed in, a programming language or computer program. A “Programming Language” and “Computer Program” is any language used to specify instruction to a computer, and includes (but is not limited to) these languages and their derivatives: Assembler, Basic, Batch files, BCPL, C, C+, C++, Delphi, Fortran, Java, JavaScript, Machine code, operating system command language, Pascal, Perl, PL1, scripting languages, Visual Basic, meta-languages which themselves specify programs, and all first, second, third, fourth, and fifth generation computer languages. Also included are database and other data schemas, and any other meta-languages. For the purpose of this definition, no distinction is made between languages which are interpreted, compiled, or use both compiled and interpreted approaches. For the purpose of this definition, no distinction is made between compiled and source versions of a program. Thus, reference to a program, where the programming language could exist in more than one state (such as source, compiled, object, or linked) is a reference to any and all such states. The definition also encompasses the actual instructions and the intent of those instructions.

Any of the herein described methods, programs, algorithms or codes may be contained on one or more machine-readable media or memory. The term “memory” may include a mechanism that provides (e.g., stores and/or transmits) information in a form readable by a machine such a processor, computer, or a digital processing devices. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

Sensor 306 may gather data about the fluids (e.g., liquid or gas) passing through the chest tube 108. Sensor 306 may be a pressure senor for monitoring the fluid pressure within the chest tube 108. Pressure sensor 306 will function the same or similar to the pressure sensor 260 as described above. Additionally, sensor 306 may be capable to monitor the fluid flow rate. Flow rate sensor 306 will function the same or similar to the flow rate sensor 260 as described above. Sensor 306 may be capable of monitor other characteristics of the fluids passing through the chest tube 108. Sensor 306 may be able to differentiate the types of fluids exiting from the patient's chest cavity. Also, sensor 306 may be able to identify the presence and volume or concentration of blood, if any, in the contents of the chest tube 108. When sensor 306 is gathering information regarding the presence of blood, sensor 306 will function the same or similar to the sensor 266. Sensor 306 may have the capabilities of measuring the pH level of the fluids, as well as the ability to detect the presence of CO₂ . When sensor 306 is gathering information regarding the pH level and/or CO₂ level, sensor 306 will function the same or similar to sensor 262 and/or sensor 264. Also, if blood is present in the fluid within the chest tube 108, sensor 306 may have the ability to detect the glucose levels. Additionally, sensor 306 may have the capability of identifying the color and turbidity of the fluids within the chest tube 108. In gathering information regarding the color and turbidity of the fluids, sensor 306 will function the same or similar to sensor 266. Measuring the pH and glucose levels and detecting the color and turbidity of the fluids within the chest tube 108 may enable the clinician to detect the presence of an infection. The detection of carbon dioxide may indicate an air leak in the patient's lung. Sensor unit 300 communicates with sensor 306 and display unit 400 to analyze the data collected by sensor 306 and uses algorithms to determine if any of the collected data were outside of the normal range and if so, sends a communication to the appropriate clinician.

Sensor unit 300 may communicate with the motorized chest drainage system 100 via any conventional wireless technology. Also, the sensor unit 300 may communicate with the motorized chest drainage system 100 through a wired port on the sensor unit 300, such as a USB port or any similar technology. The sensor unit 300 may also communicate with a mobile device, such as a mobile phone or pager. This communication may be accomplished by using any conventional wireless technology. The clinician may preselect the information the sensor unit 300 will communicate with the motorized chest drainage system 100 and/or the mobile device.

The sensor unit 300 can work in tandem with motorized chest drainage system 100 or can be placed on a non-smart chest drainage system. Also, the motorized chest drainage system 100 can fully function without the inclusion of sensor unit 300. When sensor 300 is included in the motorized chest drainage system 100, sensor 300 may augment the function of the motorized chest drainage system 100.

With reference to FIGS. 1-6, use of the motorized chest drainage system 100 is discussed. During the course of a thoracic surgical procedure or in a situation where fluids (e.g., liquid or gas) need to be removed from a patient's thoracic cavity, a clinician inserts the articulating tip 111 of the motorized chest drainage system 100 through an opening in tissue (e.g., an incision) into the pleural cavity. The articulating tip 111 may be inserted anteriorly, posteriorly, or laterally into the pleural cavity. Once the articulating tip 111 is placed within the pleural cavity, the clinician may adjust the placement of the articulating tip 111 by remotely controlling the actuation assemblies 130, selecting a predetermined oscillation pattern of the articulating tip, or selecting a user defined pattern of tip movement. Prior to use, the practitioner may program a user defined pattern of tip movement in view of the procedure to be performed. Initially, the practitioner may manually control the articulating tip 111 and the removal of fluids until the patient's condition is such that automatic control of the articulating tip 111 is possible or desirable. During use, the motorized chest drainage system 100 may be temporarily suspended in order to drain collected fluids from fluid reservoir 109. Alternatively, the fluid reservoir 109 may be coupled to a drainage system allowing for continued and uninterrupted operation.

In any of the embodiments disclosed herein, the motorized chest drainage system 100 can be programmed to have an autonomous, randomly oscillating tip with intelligence. The movement of the articulable tip 111 can randomly oscillate, or follow a simple algorithm that defines a pattern, such as circular, criss-crossing movement, up and down or side to side reciprocation, or a combination of these. Thus, the motorized chest drainage system 100 can have a flexible or articulated tube that is motorized and has pre-programmed movements. Furthermore, in the presently disclosed embodiments, the sensor positioned on the suctioning tip may be a pressure sensor, a flow rate sensor, a pH sensor, a gas sensor, or a fluid content sensor. The pressure sensor may be used to determine the pressure of the fluids in the pleural space and can detect leaks in the lung.

A gas sensor can be used to determine if there is a leak by detecting the presence or quantity of CO₂, O₂, or other gases. A fluid content sensor can detect the presence of blood or another fluid, and the pH sensor can be used to identify a possible infection.

Pressure changes at the articulable tip 111 can also indicate the presence of a blockage, or that the articulable tip 111 has suctioned tissue or other particulate matter in the pleural space. In the event that the pressure sensor indicates a blockage or that the articulable tip 111 is stuck on tissue, the motorized chest drainage system 100 can be programmed to “puff” and blow air, CO₂ or another biocompatible gas or liquid through the chest tube 108 and then to move the articulable tip 111 in an opposite direction. In any of the embodiments disclosed herein, the motorized chest drainage system 100 can be programmed so that after detecting an undesirable reading from the sensors 260, 262, 264, and 266, the movement of the articulable tip 111 is changed, reversed in direction, or modified. In addition, a user of the motorized chest drainage system 100 can manually, or by interaction with an interface on the housing 140, change the movement of the articulable tip 111. In another example, the motorized chest drainage system 100 can be programmed so that the movement of the articulable tip 111 is automatically changed in response to an indication that movement is resisted. Such indication can come from movement and positioning sensors on the articulable tip 111.

Smart programming, or artificial intelligence, can be included in the motorized chest drainage system 100, in any of the embodiments. For example, the motorized chest drainage system 100 can track how often the articulable tip 111 becomes blocked or stuck, and establish a different pattern of movement on that basis. The motorized chest drainage system 100 can provide a report to the user, so that the user can understand that certain patterns of movement are undesirable and interact with the motorized chest drainage system 100 to make changes.

The entire contents of U.S. Patent Application Serial No. 62/275,829 filed Jan. 7, 2016, is hereby incorporated by reference herein.

It is contemplated that the motorized chest drainage system 100 may use wireless communications to send configurable reports or notifications on the patient's condition to mobile communications devices, such as mobile phones or pagers, as carried by a clinician who may be monitoring the patient's condition. Such wireless notifications may alert a clinician that the chest tube 108 has become blocked, or disconnected from the patient, the presence of a possible infection, or a leak coming from the patient's lungs.

It will be understood that various modifications may be made to the embodiments disclosed herein. Therefore, the above description should not be construed as limiting, but merely as exemplifications of embodiments. Those skilled in the art will envision other modifications within the scope and spirit of the present disclosure.

What is claimed is:

1. A chest drainage system, comprising:
a flexible tube having proximal and distal ends;
a tip positioned at the distal end of the flexible tube;
a de-clogging mechanism operatively coupled to the flexible tube;
an articulation assembly operatively coupled with the flexible tube, the articulation assembly is adapted to articulate the tip; and
a control assembly which is operated by a motor, the control assembly operatively coupled with the articulation assembly.

2. The chest drainage system of claim 1, further including a suction source including a sensor in operative communication with the suction source to collect data on suction pressure, fluid flow rate, or volume of fluid, or content type or any combination thereof.

3. The chest drainage system of claim 2, further including a data processor to evaluate data collected by the sensor.

4. The chest drainage system of claim 2, further including a fluid reservoir.

5. The chest drainage system of claim 4, further having a case attached to the proximal end of the flexible tube, the case housing the suction source, the sensor, the data processor, the fluid reservoir and the control assembly.

6. The chest drainage system of claim 5, wherein the case includes a display for displaying data collected by the data processor.

7. The chest drainage system of claim 5, wherein the case also includes a power outlet or a battery, or both.

8. The chest drainage system of claim 5, wherein the case also includes a control for the articulation assembly and a motor control.

9. The chest drainage system of claim 1, further including a de-clogging mechanism.

10. The chest drainage system of claim 1, wherein the articulation assembly is remotely operated.

11. The chest drainage system of claim 1, wherein the articulation assembly is programmed to have a set oscillation pattern.

12. The chest drainage system of claim 1, wherein the articulation assembly is programmed to have a user defined pattern.

13. The chest drainage system of claim 1, further including a detachable sensor unit disposed in-line with the flexible tube.

14. The chest drainage system of claim 13, wherein the detachable sensor unit includes a sensor to gather data about the pressure, flow rate, pH, presence of blood, carbon dioxide levels and glucose levels, or any combination thereof.

15. The chest drainage system of claim 14, wherein the detachable sensor unit includes a display system, the display system includes a data processor to evaluate the date collected by the sensor and a display unit to display data collected by the sensor.

16. The chest drainage system of claim 15, wherein the detachable sensor unit wirelessly communicates the data collected by the sensor to the chest drainage system, a mobile device, or any combination thereof.

17. The chest drainage system of claim 13, wherein the detachable sensor unit includes a battery which provides power to the detachable sensor unit. 27. A method for placing a chest drainage system, comprising:
a flexible tube having proximal and distal ends;
a tip positioned at the distal end of the tube;
an articulation assembly operatively coupled with the flexible tube, the articulation assembly is adapted to articulate the tip;
a control assembly which is operated by a motor, the control assembly operatively coupled with the articulation assembly; and
a suction source including a sensor in operative communication with the suction source to collect data on suction pressure, fluid flow rate, or content type or any combination thereof;
inserting the tip within a pleural cavity;
operating the articulation assembly to adjust the tip within the pleural cavity to secure a desired anatomical location; and
adjusting the tip within the pleural cavity after a pleural effusion is detected.
